(19) United States
(12) Patent Application Publication
(10) Pub. No.: US 2018/0336963 A1
(43) Pub. Date: Nov. 22, 2018

(54) Meaningfulness Measure

(71) Applicants: David Burke, Halifax (CA); Dan LeBlanc, Halifax (CA)

(72) Inventors: David Burke, Halifax (CA); Dan LeBlanc, Halifax (CA)

(21) Appl. No.: 15/982,822

(22) Filed: May 17, 2018

Related U.S. Application Data
(60) Provisional application No. 62/507,700, filed on May 17, 2017.

(51) Int. Cl. G16H 20/70; G16H 40/67; G16H 50/20

(52) U.S. Cl. CPC G16H 20/70; G16H 50/20; G16H 40/67

(57) ABSTRACT

Techniques are provided which may allow meaningful social engagements to be measured, tracked, and enhanced to allow for enhanced care for each individual receiving care in a facility or home. A combination of type, frequency proximity, and duration, plus key profile traits may factor into an overall number to rate meaningfulness of social engagements for each individual.

### FIELD

This disclosure relates to a meaningfulness measure.

### BACKGROUND

Meaningful social engagement is one of the most powerful predictors of positive aging, longevity, and quality of life. Care providers have no way to measure, encourage or predict meaningful social engagement of individuals receiving care in a facility or home. The result is loneliness at epidemic levels resulting in memory loss, depression, anxiety and even death.

### SUMMARY

The instant application discloses, among other things, techniques to allow meaningful social engagements to be measured and tracked, providing feedback to allow for enhanced care for each individual who may be receiving services in a facility or home.

In one embodiment, wearable devices (wearables), such as wristbands, may be worn by residents, staff members, or visitors to a care facility or home. The wearables may provide information about proximity to other wearables, so that type, frequency, proximity, and duration of meaningful social engagements between people may be measured, tracked, and enhanced. This may provide insight into the quality of engagements a cared-for individual has with other people, with the goal of enhancing mental wellbeing.

### BRIEF DESCRIPTION OF THE DRAWINGS

The present description may be better understood from the following detailed description read in light of the appended drawings, wherein:

FIG. 1 is an illustration of a resident meeting with an interactor, according to one embodiment.

FIG. 2 is a table showing sample values for a type of interactor, according to one embodiment.

FIG. 3 is a table showing sample values for a frequency of meaningful social engagement, according to one embodiment.

FIG. 4 is a table showing sample values for a duration of a meaningful social engagement, according to one embodiment.

FIG. 5 is a table showing sample values for a proximity of a meaningful social engagement, according to one embodiment.

FIG. 6 is a table showing sample values for profile traits of a resident, according to one embodiment.

FIG. 7 illustrates a system that may support a Meaningfulness Measure, according to one embodiment.

FIG. 8 is an example of an operator dashboard of a Meaningfulness Measure user interface, according to one embodiment.

FIG. 9 is an example of a resident dashboard of a Meaningfulness Measure user interface, according to one embodiment.

FIG. 10 is a block diagram illustrating an example of a system capable of supporting a Meaningfulness Measure, according to one embodiment.

FIG. 11 is a component diagram of a computing device which may support a Meaningfulness Measure process according to one embodiment.

### DESCRIPTION

A more particular description of certain embodiments of meaningfulness measure may be had by references to the embodiments shown in the drawings that form a part of this specification, in which like numerals represent like objects.

Due to a lack of meaningful social engagement, individuals, for example, a senior citizen receiving care at a facility or living at home, or individuals of other ages or demographics, are at risk to experience loneliness, resulting in negative health outcomes and even death. Meaningfulness Measure may provide techniques to allow meaningful social engagements to be measured and tracked, providing feedback to allow for enhanced care for each individual.

For example, wearables may be worn by residents, staff members, or visitors. The wearables may provide information about proximity to other wearables, so that type, frequency, proximity, and duration of meaningful social engagements between people may be measured, tracked, and enhanced. This may provide insight into the quality of engagements a resident has with other people.

Other types of devices may be used to provide information about type, frequency, proximity, and duration of meaningful social engagements.

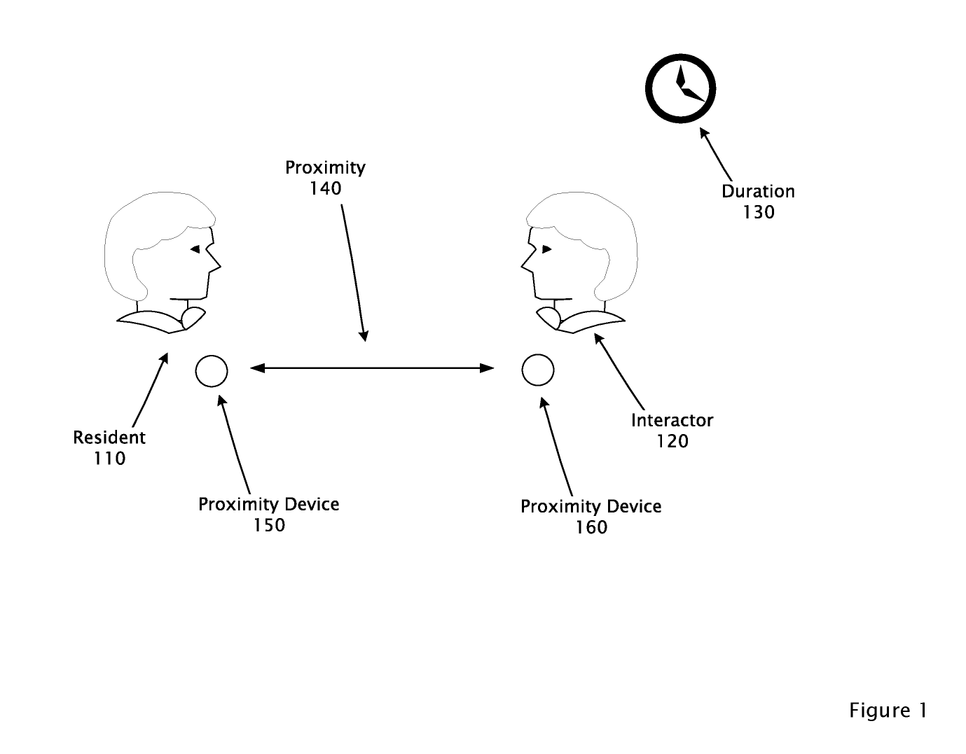

FIG. 1 is an illustration of Resident 110 meeting with Interactor 120. Resident 110 may be any individual who is receiving, or in need of receiving, care in a facility or home. For example, Resident 110 may be a senior citizen or person of any age residing in a care facility or at home. Interactor 120 may be, for example, a passerby, a stranger, an acquaintance, a colleague, care staff, a friend, a member of an extended family, a tablemate, a partner, a member of the core family, or someone from the community. Proximity 140 may be a distance between Resident 110 and Interactor 120. Each of Proximity Device 150 and Proximity Device 160 may be, for example, a device that allows a determination of the distance between the two devices.

Proximity 140 may be a factor affecting meaningfulness of a meaningful social engagement. For example, Proximity 140 may, for example, be grouped into categories, which may indicate levels of intimacy.

Duration 130 may indicate an amount of time a meaningful social engagement lasts. Duration 130 may be another factor affecting meaningfulness of a social engagement.

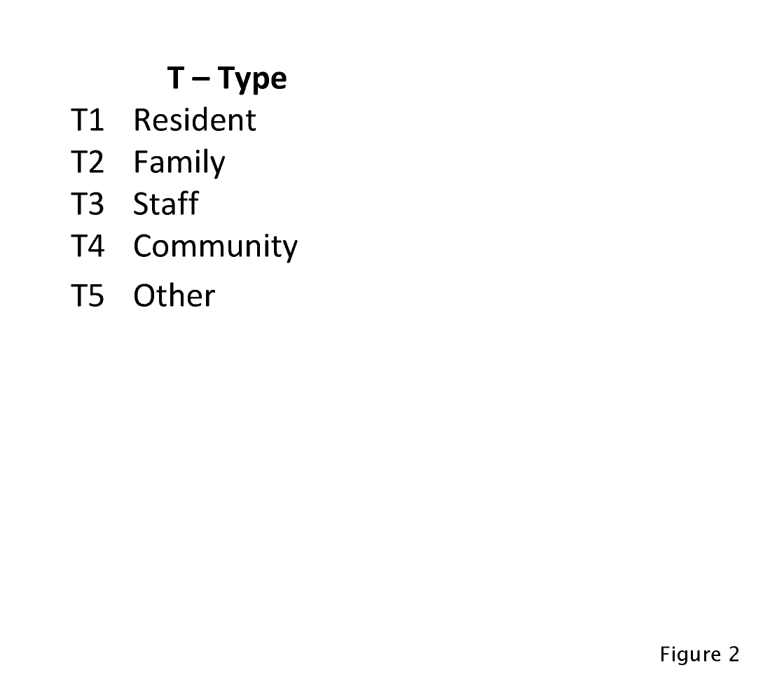

FIG. 2 is a table showing sample values for a type of interactor. An identity of Interactor 120 may be determined by a wearable device, face recognition, gait analysis, a manual analysis, or any another technique. One having skill in the art will recognize that many different devices and techniques may be used to identify a person.

For example, a social engagement with a member of a Resident 110's Family may have more meaning for Resident 110 than a social engagement with a stranger. For multiple meaningful social engagements, summation may be used for Frequency (F), and average may be used for Duration (D) and Proximity (P).

Values for Type may be personalized for each resident. For example, family members may be not all be equally important for Resident 110 to see. Some members may have a higher value placed on them than others.

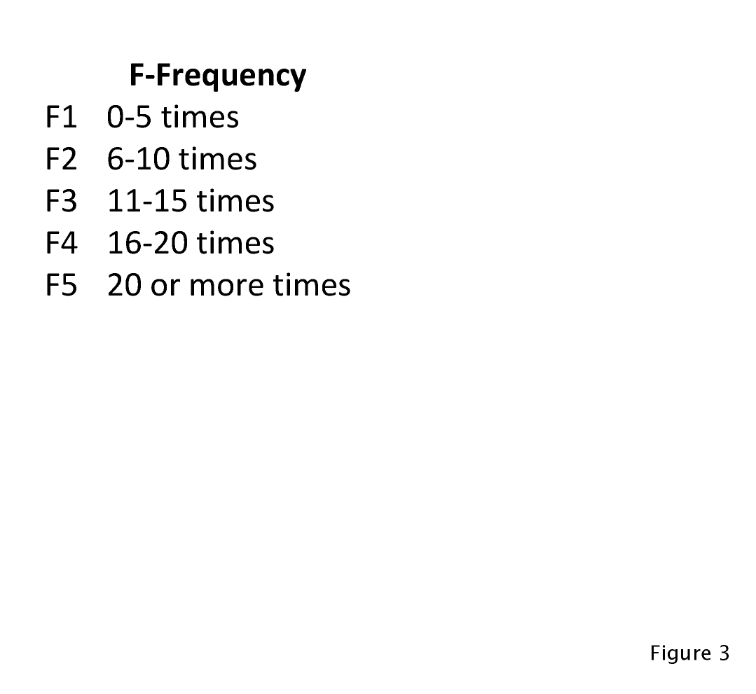

FIG. 3 is a table showing sample values for a Frequency (F) of engagement. Frequency of engagements may measure how often Resident 110 engages with a particular Interactor 120.

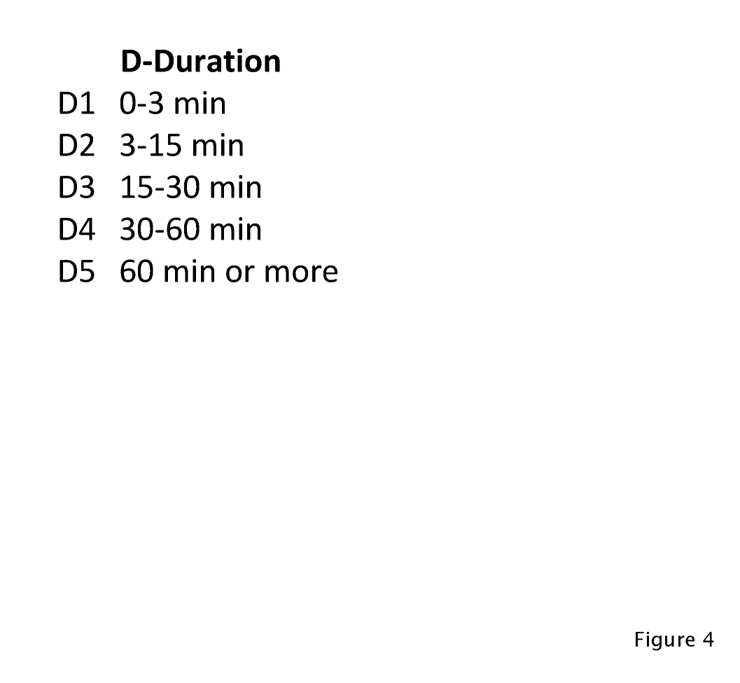

FIG. 4 is a table showing sample values for a Duration (D) of a meaningful social engagement. Longer durations may indicate a higher quality engagement or different meaningfulness.

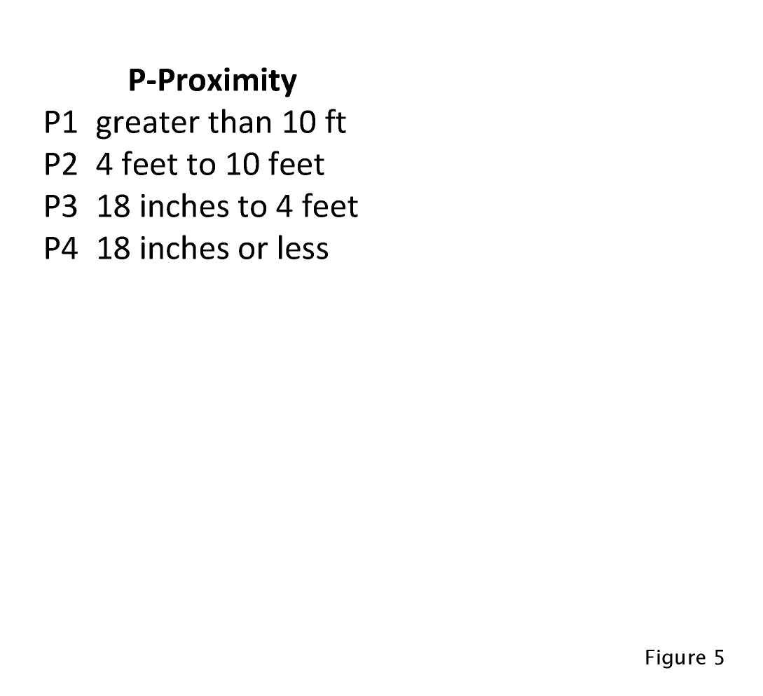

FIG. 5 is a table showing sample values for a Proximity (P) of a meaningful social engagement. Each of Proximity Device 150 and Proximity Device 160 may be, for example, a device that allows a determination of a distance between the two devices. They may have a form factor of a bracelet, a necklace, smart glasses, or other wearables, for example. In another embodiment, Proximity 140 may be determined, for example, by cameras, floor sensors, heat sensors, or other sensors in an area. One having skill in the art will recognize that many different devices and techniques may be used to measure Proximity 140.

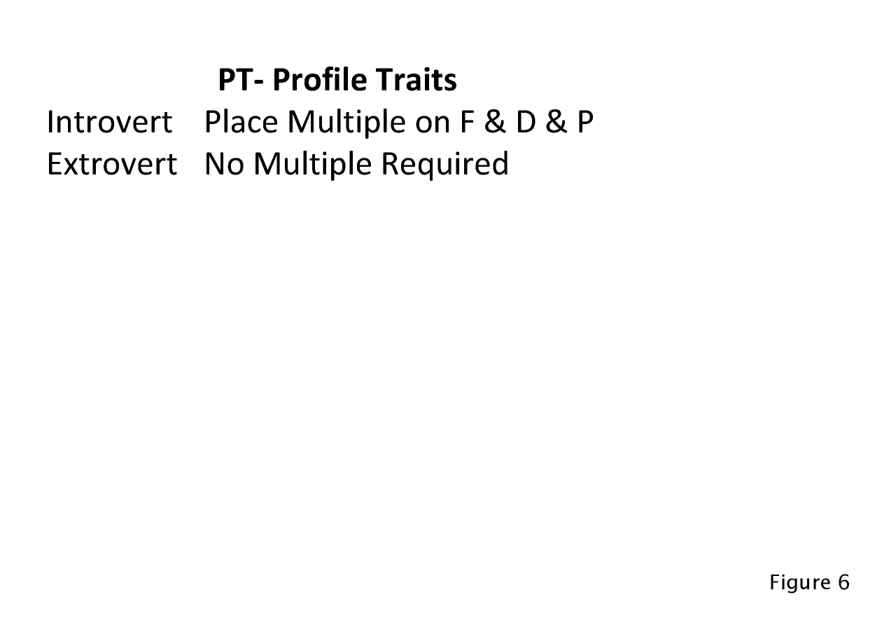

FIG. 6 is a table showing sample values for profile traits of a resident. Profile traits (PT) may include personality traits, for example, introvert or extrovert, and may also include traits such as risk for wandering, risk of roaming, risk of falls, or items unique to a person's specific care plan. An Introvert may not require the same Frequency (F), Duration (D), or Proximity (P) as an Extrovert. To compensate for this difference in required engagements, multipliers may be applied to meaningful social engagements for an Introvert. For example, the values of F, D and P may be doubled when calculating a Meaningfulness Measure for an Introvert. In another embodiment, different multipliers may be used for different factors. For example, F may be multiplied by 1.5, D by 2, and P by 2.5. In yet another embodiment, the multipliers may be customized for each Resident.

An Introvert may have different needs or desires which may influence the Meaning of a social engagement. For example, a social engagement with a member of a Resident's 110 Family may have more meaning for Resident 110 than a social engagement with a stranger. For multiple meaningful social engagements, summation may be used for Frequency (F), and average may be used for Duration (D) and Proximity (P).

With these various metrics in place, a Meaningfulness Measure may be calculated. In one embodiment, a Global Meaningfulness Measure may be reflected in the following formula: (T+F+P+D) PT=M (1 through 4). For example, Mary, a resident, may have the following meaningful social engagements metrics during a week:

TABLE 1

| Interactor | Metrics |
|---|---|
| Thelma (Resident Friend) | (T1 + F4 + P3 + D3) PT1 = M3 |
| Paul (Recreation Care Staff) | T3 + F5 + D2 + P4 = M4 |
| Karen (Cleaning Staff) | T3 + F3 + D2 + P2 = M3 |
| Connie (Family) | T2 + F1 + D3 + P3 = M3 |
| Tim (Resident Friend) | T1 + F1 + D1 + P2 = M2 |

Based on this example, Mary's Meaningfulness Measure for the week may be calculated as M3. Each factor may be considered, so that in this example, Mary's engagement with Thelma give the numbers (1+4+3+3)/4, which gives 11/4, rounded to 3 to give M3. For Mary's engagement with Paul, we get (3+5+2+4)/4=14/4, rounded to a rating of M4.

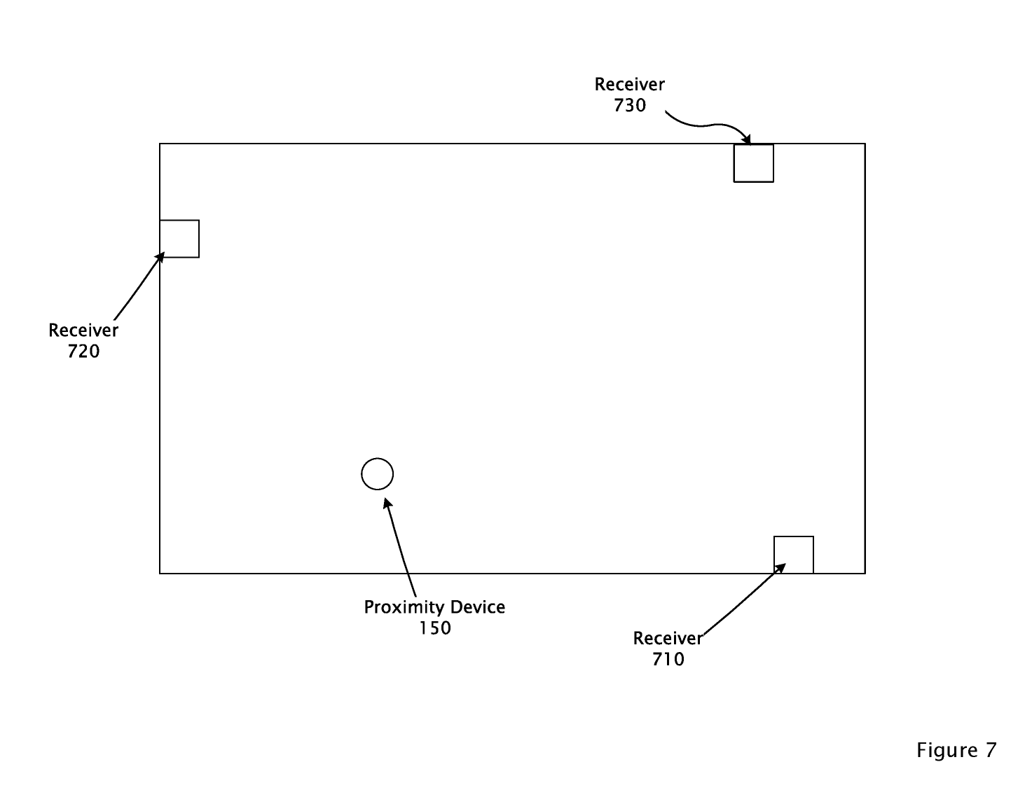

FIG. 7 illustrates a system that may support some aspects of a Meaningfulness Measure. Receivers 710, 720, 730 may receive signals from Proximity Device 150, including, for example, an identification of Proximity Device 150 and a time stamp. Master Receiver 810 may collect data from each Receiver 710, 720, 730, and may send this collected data to Server 850. In another embodiment, each Receiver 710, 720, 730 may send this data directly to Server 850.

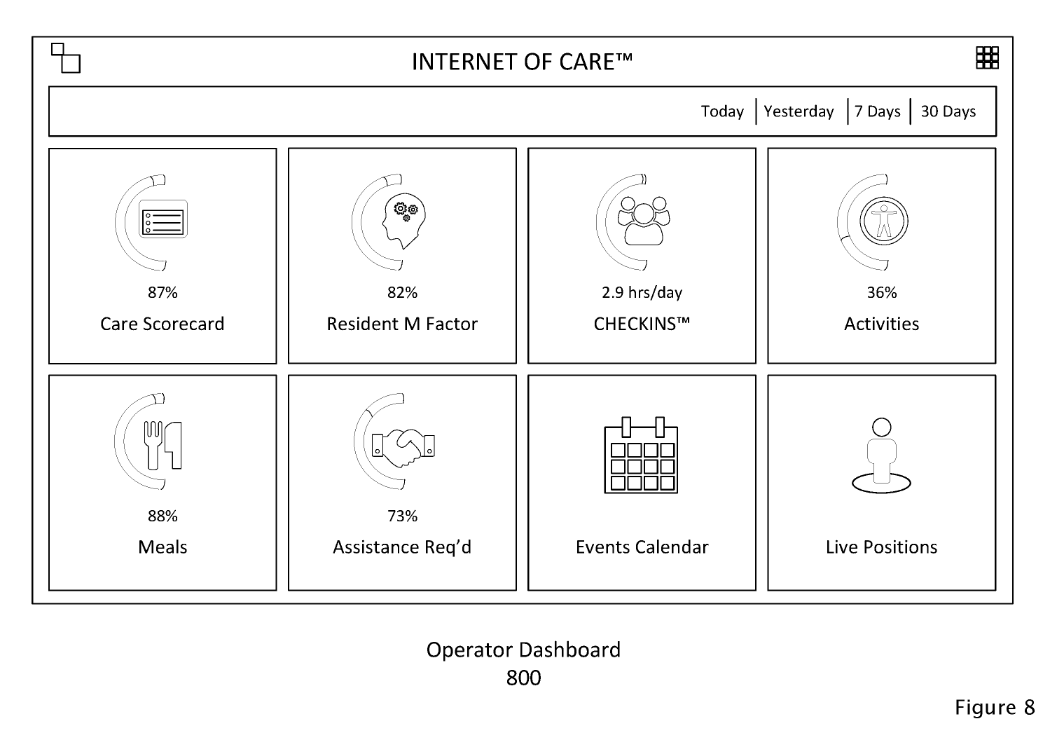

FIG. 8 is an example of an operator dashboard of a Meaningfulness Measure user interface, according to one embodiment. A Meaningfulness Measure system may be executed on a software application, for example, a native or web-based application for a desktop, laptop, or mobile device. After verifying an account, a user may log in to the Meaningfulness Measure system and view a user interface which includes a dashboard. The user may be an operator of a care facility or home, a caretaker, resident, family member, or friend. In this example, Operator Dashboard 800 may provide a dashboard tailored to an operator of a care facility or home.

Operator Dashboard 800 may display a plurality of squares, or bricks, displaying high-level information and graphics. The information and graphics may provide an overview of metrics for various types of interactions in which meaningful social engagement has taken place by residents at the facility or home.

The Global Meaningfulness Measure, reflected in the formula (T+F+P+D) PT=M (1 through 4), may be used to categorize a type of meaningful social engagement that takes place during each interaction between Resident 110 and other residents, care staff, volunteers, family, and friends. In one embodiment, an interaction may be referred to as a “CheckIn.” A CheckIn may comprise a specific engagement involving at least 4 different data points. For example, a CheckIn may be registered if an engagement between a Resident 110 and Interactor 120, or between Resident 110 and another resident, is within a specific proximity for at least 30 seconds. Distinguishing between an interaction and CheckIn may relate to measuring a meaningfulness of social engagements, which may ultimately impact a Resident 110's overall M Factor.

Care facilities are governed by many regulations but are guided by very few standards. Meaningfulness Measure may help establish measurable standards, for example, by recommending that each Resident 110 have at least 3 or more hours of CheckIns on a daily basis.

A Resident 110's M Factor may measure that individual's overall mental health or wellness. The Global Meaningfulness Measure may be used to calculate an individual's M Factor. The Global Meaningfulness Measure formula may include the following inputs:

Individual's M Factor=CheckIns, Meals, Activities, Requests for Assistance Required.

The Global Meaningfulness Measure may comprise an essential factor in determining the individual's M Factor. The M Factor may be calculated using key inputs from all the meaningfulness measure product features. A high (blue) M Factor may indicate an excellent level of mental wellness. A low (red) M Factor may indicate a resident whose overall mental wellness is at risk.

Metrics may be factored into a percentile or displayed using charts, graphs, colors, or labels, for example. In one embodiment, some bricks may have a dial or gauge, which may comprise semi-circle, located to a left of a center image on the brick, with colors indicating levels of meaningfulness, for example, Excellent, Great, OK, At Risk, and blue may be used to represent a highest value of meaningful social engagement.

In this example, Operator Dashboard 800 may display bricks which provide a high-level overview of various types of interactions or CheckIns in which meaningful social engagement is measured at a facility or home. For example, a Meals brick may provide metrics indicative of both social engagement and nutrition. The Meals brick may capture meal attendance in a dining room and meals received in individual suits. Attending meals in a dining room may provide a specific type of social engagement which contributes to a resident's overall M Factor. In this example, a Meals brick may show that 88% of scheduled meal times have been attended by residents during a given day, week, or month, whether in a dining room geofence or in an individual suite. When the operator clicks on a brick, the operator may see more detailed information, for example, graphical and table-based reports on each Resident 110 for different time periods. In one embodiment, a Resident 110 who remains in a geofence such as dining area for longer than 5 minutes during a scheduled serving of meals may receive a meal “CheckIn.” The meal CheckIn may serve as one of many factors used to assess a resident's level of meaningful social engagement, mental health, and overall well-being. Operator Dashboard 800 may help the operator identify which are at risk of improper nutrition or loneliness, and in need of enhanced care.

An Assistance Required brick may provide information about what type of assistance a Resident 110 has requested from a caregiver during a time period. The brick may also provide information regarding a quality or promptness of response by the caregiver. Each wearable, or “liveable” device, for example, a wristband, may include a red button to request assistance. When a button is pressed, a team member may be required to approach a resident and be within a specific proximity for at least 5 seconds to close out the request. This may be designed to ensure a resident is meaningfully engaged to determine a nature of their request. In some instances, a request may be completed “on the spot,” or immediately. In other instances, a staff member could need other resources and may have to return to complete the request. It is possible that a request for assistance was closed, but an interaction did not last long enough to qualify as a CheckIn.

A Meaningfulness Measure alert monitoring system may provide a Nurse Call or wandering (risk for elopement) functionality, or notifications for residents at risk for Loneliness and Roaming, which may be related to “sundowning syndrome,” for example.

Friend CheckIns may comprise a 1:1 engagement involving a Resident 110. An engagement may only register as a CheckIn if it takes place within a specific proximity and lasts for at least 30 seconds, for example.

The Meaningfulness Measure system may be configured to distinguish between personal and social CheckIns. Distinguishing between an Interaction and CheckIn may relate to measuring a meaningfulness of social engagements which will ultimately impact a resident's overall M Factor. A number or duration of CheckIns may be a set goal for each resident. A scale or dial may turn a certain color, for example, green, when 3 or more hours of CheckIns are achieved (for example, 50%).

Staff CheckIns may comprise a 1:1 engagement involving Care Staff and a Resident. An engagement may only be registered as a CheckIn if it takes place within a specific proximity and lasts for at least 30 seconds. These CheckIns may be further categorized based on a role of each Care Staff. Like Friend CheckIns, 1.5 hours of daily Staff CheckIns may be selected as a goal for each resident.

An Events brick may comprise a calendar of facility events to help facilitate conversations and encourage physical and social activity. A Live Positions brick may provide locations of each Resident 110 in a care facility or home, based on a location of a proximity device worn by a resident.

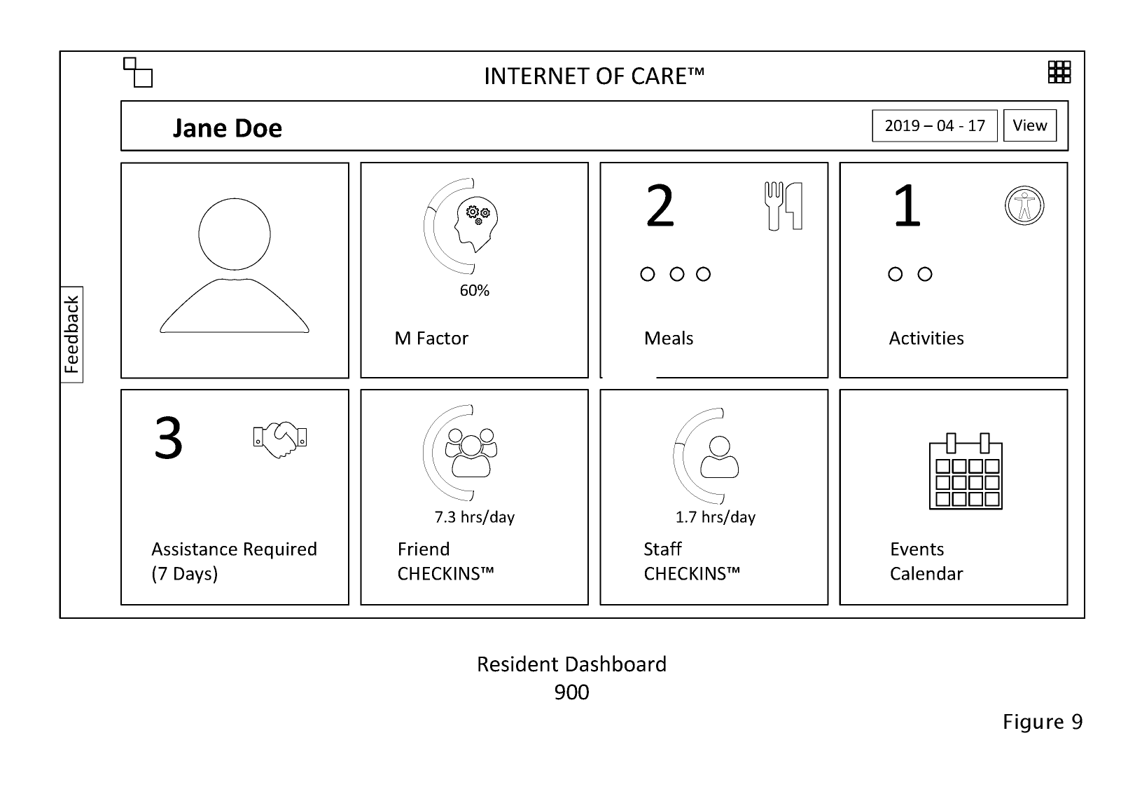

FIG. 9 is an example of a resident dashboard of a Meaningfulness Measure user interface, according to one embodiment. After verifying an account, a user, such as Resident 110, family member, friend, or concerned loved one, may log in to the Meaningfulness Measure system and view Resident Dashboard 900. The dashboard may include various bricks displaying high-level information about Resident 110's meaningful social engagement in a facility or at home. When clicked, each brick may display a profile of Resident 110 and include detailed information about the resident's interactions, or CheckIns.

Resident Dashboard 900 may include a feedback button on a left side of the dashboard which to provide an opportunity for users to send a private message to Meaningfulness Measure developers or customer service. This may provide an easy, quick way to submit feedback to ask questions about a specific Internet of Care feature or suggest features a user might like to see in the future.

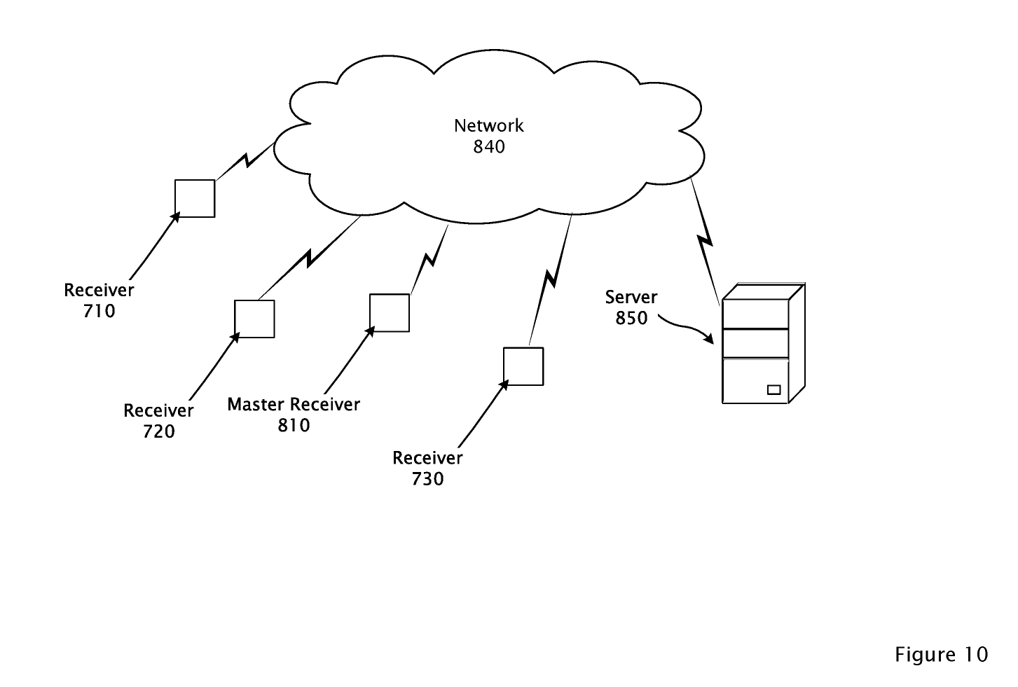

FIG. 10 is a block diagram illustrating an example of a system capable of supporting a Meaningfulness Measure, according to one embodiment. Network 840 may include Wi-Fi, cellular data access methods, such as 3G or 4GLTE, Bluetooth, Near Field Communications (NFC), the internet, local area networks, wide area networks, or any combination of these or other means of providing data transfer capabilities. In one embodiment, Network 840 may comprise Ethernet connectivity. In another embodiment, Network 840 may comprise fiber optic connections.

Receivers 710, 720, or 730 may have network capabilities to communicate with Server 850. Server 850 may include one or more computers, and may serve a number of roles. Server 850 may be conventionally constructed, or may be of a special purpose design for processing data obtained from a Meaningfulness Measure. One skilled in the art will recognize that Server 850 may be of many different designs and may have different capabilities.

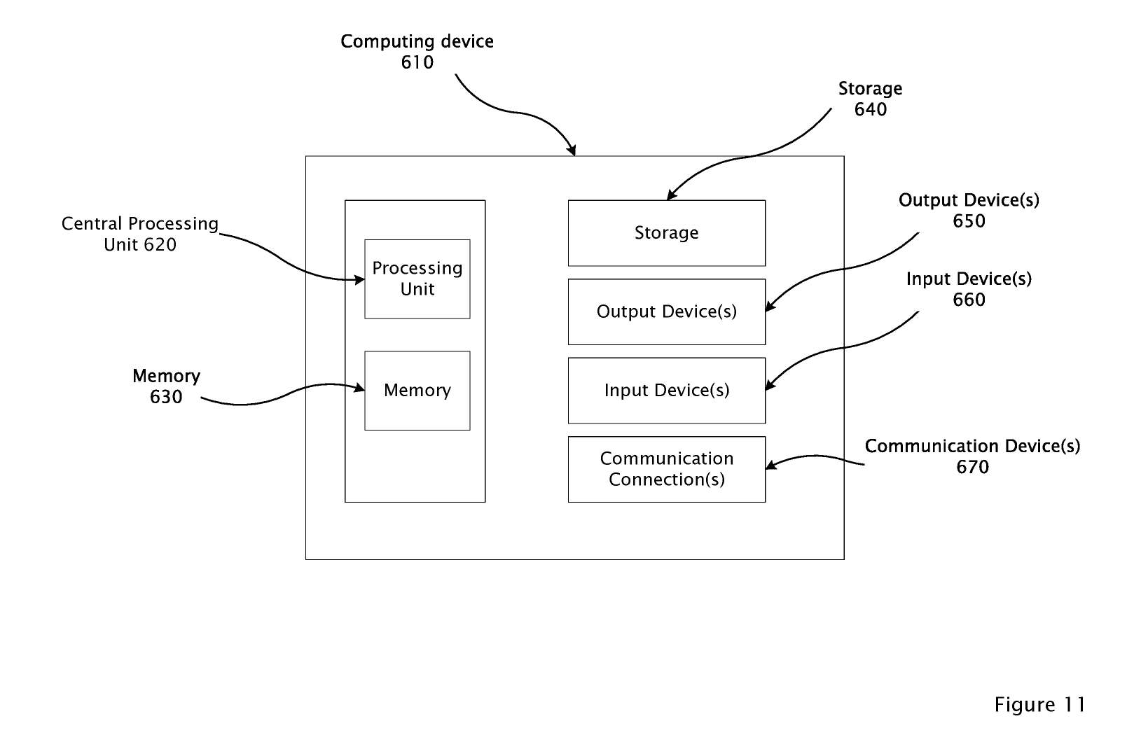

FIG. 11 is a component diagram of a computing device which may support a Meaningfulness Measure process according to one embodiment. Computing Device 610 can be utilized to implement one or more computing devices, computer processes, or software modules described herein, including, for example, but not limited to a mobile device. In one example, Computing Device 610 can be used to process calculations, execute instructions, and receive and transmit digital signals. In another example, Computing Device 610 can be utilized to process calculations, execute instructions, receive and transmit digital signals, receive and transmit search queries and hypertext, and compile computer code suitable for a mobile device. Computing Device 610 can be any general or special purpose computer now known or to become known capable of performing the steps or performing the functions described herein, either in software, hardware, firmware, or a combination thereof.

In its most basic configuration, Computing Device 610 typically includes at least one Central Processing Unit (CPU) 620 and Memory 630. Depending on the exact configuration and type of Computing Device 610, Memory 630 may be volatile (such as RAM), non-volatile (such as ROM, flash memory, etc.) or some combination of the two. Additionally, Computing Device 610 may also have additional features/functionality. For example, Computing Device 610 may include multiple CPU's. The described methods may be executed in any manner by any processing unit in computing device 610. For example, the described process may be executed by both multiple CPUs in parallel.

Computing Device 610 may also include additional storage (removable or non-removable) including, but not limited to, magnetic or optical disks or tape. Such additional storage is illustrated by Storage 640. Computer readable storage media includes volatile and nonvolatile, removable and non-removable media implemented in any method or technology for storage of information such as computer-readable instructions, data structures, program modules or other data. Memory 630 and Storage 640 are all examples of computer-readable storage media. Computer readable storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can accessed by computing device 610. Any such computer-readable storage media may be part of computing device 610. But computer-readable storage media does not include transient signals.

Computing Device 610 may also contain Communications Device(s) 670 that allow the device to communicate with other devices. Communications Device(s) 670 is an example of communication media. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (RF), infrared and other wireless media. The term computer-readable media as used herein includes both computer-readable storage media and communication media. The described methods may be encoded in any computer-readable media in any form, such as data, computer-executable instructions, and the like.

Computing Device 610 may also have Input Device(s) 660 such as a keyboard, mouse, pen, voice input device, or touch input device, etc. Output Device(s) 650 such as a display, speakers, printer, etc. may also be included. All these devices are well known in the art and need not be discussed at length.

Those skilled in the art will realize that storage devices utilized to store program instructions may be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realize that by utilizing conventional techniques known to those skilled in the art that all, or a portion of the software instructions may be carried out by a dedicated circuit, such as a digital signal processor (DSP), programmable logic array, or the like.

The foregoing description of various embodiments of the invention has been presented for the purposes of illustration and description. It is not intended to be exhaustive or to limit the invention to the precise form disclosed. It is intended that the scope of the invention be limited not by this detailed description, but rather by the claims appended hereto. The above specification, examples, and data provide a complete description of the manufacture and use of the invention. Since many embodiments of the invention can be made without departing from the spirit and scope of the invention, the invention resides in the claims hereinafter appended.

1. A method of measuring meaningful engagement between individuals, comprising the steps of:
placing a tracking device on an individual or location within a facility or home, wherein at least one individual is receiving or is in need of receiving care;
receiving data from the tracking device;
analyzing the received data;
determining an action to enhance care for an individual based on the analyzed data; and
performing the action.

2. The method of claim 1, wherein the tracking device comprise a wearable.

3. The method of claim 1, wherein the tracking device is configured to provide information from the list containing a type, frequency, proximity, and duration of meaningful social engagements.

4. The method of claim 1, wherein data received from the tracking device is measured and tracked.

5. A meaningfulness measure system, comprising:
receiving data from an individual in a meaningfulness measure software program;
aggregating the received data;
comparing the aggregated data against metric goals;
outputting a summary of the individual's social engagement;
providing a recommendation for enhancement of an individual's social engagement; and
implementing the recommendation.
